(19) United States
(12) Patent Application Publication
(10) Pub. No.: US 2025/0114491 A1
(43) Pub. Date: Apr. 10, 2025

(54) ROBOT SURFACE DISINFECTION APPARATUS, ROBOT SURFACE DISINFECTION METHOD USING THE SAME AND ROBOT SURFACE DISINFECTION SYSTEM

(71) Applicant: BIODECH, Inc., Suwon-si (KR)

(72) Inventors: Jin Woo KIM, Suwon-si (KR); Man Ho HAN, Hwaseong-si (KR); Jin Nam LEE, Suwon-si (KR)

(73) Assignee: BIODECH, Inc., Suwon-si (KR)

(21) Appl. No.: 18/904,704

(22) Filed: Oct. 2, 2024

(30) Foreign Application Priority Data
Oct. 4, 2023 (KR) 10-2023-0131854

(51) Int. Cl. A61L 2/10

(52) U.S. Cl. CPC A61L 2/10; A61L 2202/14; A61L 2202/20

(57) ABSTRACT

A robot surface disinfection apparatus, configured to disinfect a surface of a robot by emitting ultraviolet light to the robot, is provided. The apparatus includes a base positioned at a predetermined height from the ground, a disinfection module formed to be movable up and down between the ground and the base, and a lifting module connected to the base and the disinfection module, respectively, and configured to lift the disinfection module. The disinfection module includes a hollow frame provided with a hollow portion that is open vertically, and an ultraviolet light generating device arranged along a circumference of the hollow frame, and configured to emit ultraviolet light into the hollow portion to disinfect a surface of a robot positioned in the hollow portion.

### CROSS REFERENCE TO RELATED APPLICATION

The present application claims priority to Korean Patent Application No. 10-2023-0131854, filed on Oct. 4, 2023, the entire contents of which are incorporated herein for all purposes by this reference.

### BACKGROUND

### Field

The present invention relates to a robot surface disinfection apparatus configured to disinfect a surface of a robot by emitting ultraviolet light to the robot, a robot surface disinfection method using the same, and a robot surface disinfection system.

### Description of the Related Art

With robot technology having advanced, the development of robots with an autonomous driving function is also in progress. Representatively, robots with an autonomous driving function include robot vacuum cleaners and logistics robots. Additionally, technologies for mobile robots that perform disinfection functions while driving through a specific space have been disclosed, as described in U.S. Patent Application Publication Document (US 2022/0080074 A1, Mar. 17, 2022) and U.S. Patent Application Publication Document (US 2022/0143249 A1, May 12, 2022).

Robots capable of autonomous driving may be used in a variety of places, such as homes, hospitals, and industrial facilities, and their demand is expected to increase in the future. Meanwhile, for these driving robots or mobile robots having other ways of driving, it is necessary to treat to disinfect pathogens present on the surface of the robot to prevent infection or transmission of disease through the mobile robot.

Accordingly, among various types of robots, it may be considered to develop a disinfection device that can perform disinfection treatments on the surface of a robot, particularly in a manner that is more suitable for the movement-related features of mobile robots.

### DOCUMENTS OF RELATED ART

Patent Document 1: US 2022/0080074 A1
Patent Document 2: US 2022/0143249 A1

### SUMMARY

The present invention is directed to providing a robot surface disinfection apparatus, a robot surface disinfection method using the same, and a robot surface disinfection system that are capable of more effectively performing disinfection of a surface of a robot, including a robot that is movable.

To achieve the object of the present invention, there is provided a robot surface disinfection apparatus according to an embodiment of the present invention. The robot surface disinfection apparatus may include a base positioned at a predetermined height from the ground, a disinfection module formed to be movable up and down between the ground and the base, and a lifting module connected to the base and the disinfection module, respectively, and configured to lift the disinfection module, in which the disinfection module may include a hollow frame provided with a hollow portion that is open vertically, and an ultraviolet light generating device arranged along a circumference of the hollow frame, and configured to emit ultraviolet light into the hollow portion to disinfect a surface of a robot positioned in the hollow portion, and when the robot drives on the ground and moves to a position corresponding to the hollow portion, the disinfection module may be positioned in a state elevated to a height of the robot or higher by the lifting module so that the disinfection module does not act as an obstacle to the movement of the robot, and when the robot enters underneath the elevated disinfection module and moves to a position corresponding to the hollow portion, the disinfection module may be lowered by the lifting module to proceed to disinfect the robot in a state disposed to surround the robot.

According to an example related to the present invention, the disinfection module may be configured to continuously emit ultraviolet light at different heights between an upper end and a lower end of the robot while being lifted by the lifting module in a state in which the robot is positioned in the hollow portion.

According to an example related to the present invention, the disinfection module may further include robot detection sensors disposed on the hollow frame facing each other and configured to detect whether the robot is positioned in the hollow portion, and the ultraviolet light generating device may be configured to be driven when the robot is detected by the robot detection sensors and not driven when the robot is not detected by the robot detection sensors.

According to an example related to the present invention, the disinfection module may further include a ground detection sensor installed at a bottom portion of the hollow frame facing the ground, and the ultraviolet light generating device may be configured to be not driven when the ground is detected by the ground detection sensor.

According to an example related to the present invention, the lifting module may be configured to stop a lowering operation when the ground is detected by the ground detection sensor.

According to an example related to the present invention, the lifting module may include a bobbin rotatably installed on the base and provided with a plurality of wire windings for each height, a drive motor formed to rotate the bobbin, guide bars installed at a plurality of places of the base, and a plurality of wires each wound on the plurality of wire windings and connected to the hollow frame through the guide bars, and the plurality of wires may be configured to be unwound from the plurality of wire windings or wound on the plurality of wire windings by forward or reverse rotation of the drive motor to lift the disinfection module.

According to an example related to the present invention, the lifting module may further include a multi-stage telescopic cylinder connected to the base and the hollow frame, respectively, and formed to surround the wire therein and extendable or contractable by length adjustment of the wire.

According to an example related to the present invention, the lifting module may further include a connecting frame configured to connect the guide bars to each other to fix relative positions of the guide bars with respect to each other, a rail portion formed to extend in an intersecting form to guide movement of the guide bars so that the guide bars are movable in a first direction and a second direction that are perpendicular to each other, and a drive unit configured to move the connecting frame in the first direction or the second direction, and the wire may pass through the guide bars and the rail portion to be connected to the hollow frame.

According to an example related to the present invention, the rail portion may include a communication hole configured for the wire to pass through, and a guide groove formed on both sides of the communication hole, enabling rolling contact of balls provided at a lower portion of the guide bar.

According to an example related to the present invention, a battery may be installed in the hollow frame, configured to supply power to the ultraviolet light generating device, and a wireless charger may be installed in the base, configured to wirelessly charge the battery in a state in which the disinfection module is disposed adjacent to the base within a predetermined distance by the lifting module.

According to an example related to the present invention, the hollow frame may include a first frame connected to the lifting module, and a second frame disposed on an inner side of the first frame and connected to the first frame so as to be rotatable thereto around the hollow portion, and the ultraviolet light generating device may be disposed in at least one area of an inner circumference of the second frame and is configured to emit ultraviolet light toward the hollow portion inside the second frame, and when the disinfection module is disposed to surround the robot, disinfection of the robot may procced while the second frame rotates relative to the first frame around the hollow portion.

According to an example related to the present invention, a power supply rail connected to a power cable may be disposed in the form of a loop on an inner circumferential surface of the first frame, and a power receiving rail electrically connected to the ultraviolet light generating device may be disposed in the form of a loop on an outer circumferential surface of the second frame, the power receiving rail always maintaining a state of being in contact with the power supply rail and receiving power even during the relative rotation.

There is provided a robot surface disinfection method using a robot surface disinfection apparatus, according to another embodiment of the present invention. The robot surface disinfection method may include: detecting whether a robot is positioned within a predetermined area of the ground; and proceeding disinfection of a surface of the robot by driving a robot surface disinfection apparatus when the robot is detected to be positioned within the predetermined area, in which the robot surface disinfection apparatus may include: a base positioned at a predetermined height from the ground; a disinfection module formed to be movable up and down between the ground and the base; and a lifting module connected to the base and the disinfection module, respectively, and configured to lift the disinfection module, in which the disinfection module may include a hollow frame provided with a hollow portion that is open vertically; and an ultraviolet light generating device arranged along a circumference of the hollow frame, and configured to emit ultraviolet light into the hollow portion to disinfect a surface of the robot positioned in the hollow portion within the predetermined area, and when the robot drives on the ground and moves to a position corresponding to the hollow portion, the disinfection module may be positioned in a state elevated to a height of the robot or higher by the lifting module so that the disinfection module does not act as an obstacle to the movement of the robot, and when the robot enters underneath the elevated disinfection module and moves to a position corresponding to the hollow portion, the disinfection module may be lowered by the lifting module to proceed to disinfect the robot in a state disposed to surround the robot.

According to an example related to the present invention, the proceeding of disinfection of the surface of the robot may be configured to continuously emit ultraviolet light, by the disinfection module, at different heights between an upper end and a lower end of the robot while the disinfection module is being lifted by the lifting module.

According to an example related to the present invention, the robot surface disinfection method using the robot surface disinfection apparatus may further include stopping disinfection of the surface of the robot by detecting the upper end and the lower end of the robot, when the disinfection module deviates from a position between the upper end and the lower end of the robot.

There is provided a robot surface disinfection system, according to still another embodiment of the present invention. The robot surface disinfection system may include: a robot configured to enable autonomous driving; and a robot surface disinfection apparatus configured to disinfect a surface of the robot positioned within a predetermined area of the ground, in communication with the robot, in which the robot surface disinfection apparatus may include: a base positioned at a predetermined height from the ground; a disinfection module formed to be movable up and down between the ground and the base; and a lifting module connected to the base and the disinfection module, respectively, and configured to lift the disinfection module, in which the disinfection module may include a hollow frame provided with a hollow portion that is open vertically; and an ultraviolet light generating device arranged along a circumference of the hollow frame, and configured to emit ultraviolet light into the hollow portion to disinfect a surface of a robot positioned in the hollow portion, and when the robot drives on the ground and moves to a position corresponding to the hollow portion, the disinfection module may be positioned in a state elevated to a height of the robot or higher by the lifting module so that the disinfection module does not act as an obstacle to the movement of the robot, and when the robot enters underneath the elevated disinfection module and moves to a position corresponding to the hollow portion, the disinfection module may be lowered by the lifting module to proceed to disinfect the robot in a state disposed to surround the robot.

According to an example related to the present invention, in the robot surface disinfection system, the robot may further include an infrared light receiving module configured to detect an infrared signal, and the robot surface disinfection apparatus may further include an infrared light transmitting module configured to guide the robot by transmitting an infrared signal to an area wider than the predetermined area.

According to an example related to the present invention, the robot surface disinfection system may further include a sensor configured to detect whether an object around the robot surface disinfection apparatus is moving or whether a door in a room in which the robot surface disinfection apparatus is positioned is operating, and a control unit of the robot surface disinfection apparatus may be configured to control driving of the disinfection module on the basis of a result detected by the sensor.

### BRIEF DESCRIPTION OF THE DRAWINGS

FIG. 1 is a view illustrating the classification system for environmental technology

FIG. 2 is a view illustrating detailed technologies of an environmental health technology.

FIG. 3 is a perspective view of a robot surface disinfection apparatus and a robot surface disinfection system including the same, according to an embodiment of the present invention.

FIG. 4 is an enlarged perspective view illustrating a lifting module illustrated in FIG. 3.

FIG. 5 is a perspective view of an ultraviolet light generating device of a disinfection module illustrated in FIG. 3, viewed from different directions.

FIG. 6 is a perspective view viewed from a bottom surface of a disinfection module illustrated in FIG. 3.

FIG. 7 is a perspective view illustrating another example of the lifting module illustrated in FIG. 3.

FIG. 8 is a perspective view illustrating another example of the robot surface disinfection apparatus illustrated in FIG. 3.

FIG. 9 is a perspective view illustrating still another example of the robot surface disinfection apparatus illustrated in FIG. 3.

FIG. 10 is a perspective view illustrating still another example of the robot surface disinfection apparatus illustrated in FIG. 3.

FIG. 11 is a perspective view illustrating still another example of the robot surface disinfection apparatus illustrated in FIG. 3.

FIG. 12 is a flowchart of a robot surface disinfection method using the robot surface disinfection apparatus according to another embodiment of the present invention.

### DETAILED DESCRIPTION

Hereinafter, embodiments disclosed in the present specification will be described in detail with reference to the accompanying drawings. The same or similar constituent elements are assigned the same reference numerals regardless of reference numerals, and the repetitive description thereof will be omitted. The suffixes “module”, “unit”, “part”, and “portion” used to describe constituent elements in the following description are used together or interchangeably in order to facilitate the description, but the suffixes themselves do not have distinguishable meanings or functions. In addition, in the description of the embodiment disclosed in the present specification, the specific descriptions of publicly known related technologies will be omitted when it is determined that the specific descriptions may obscure the subject matter of the embodiment disclosed in the present specification. In addition, it should be interpreted that the accompanying drawings are provided only to allow those skilled in the art to easily understand the embodiments disclosed in the present specification, and the technical spirit disclosed in the present specification is not limited by the accompanying drawings, and includes all alterations, equivalents, and alternatives that are included in the spirit and the technical scope of the present invention.

The terms including ordinal numbers such as “first,” “second,” and the like may be used to describe various constituent elements, but the constituent elements are not limited by the terms. These terms are used only to distinguish one constituent element from another constituent element.

When one constituent element is described as being “coupled” or “connected” to another constituent element, it should be understood that one constituent element can be coupled or connected directly to another constituent element, and an intervening constituent element can also be present between the constituent elements. When one constituent element is described as being “coupled directly to” or “connected directly to” another constituent element, it should be understood that no intervening constituent element exists between the constituent elements.

Singular expressions include plural expressions unless clearly described as different meanings in the context.

In the present application, it should be understood that terms “including” and “having” are intended to designate the existence of characteristics, numbers, steps, operations, constituent elements, and components described in the specification or a combination thereof, and do not exclude a possibility of the existence or addition of one or more other characteristics, numbers, steps, operations, constituent elements, and components, or a combination thereof in advance.

Environmental technology is a technology that improves the self-cleaning ability of the environment and suppresses and eliminates factors that cause environmental damage to people and nature, and refers to various technologies necessary for the preservation and management of the environment, such as preventing or reducing environmental contamination in advance or restoring polluted and damaged environments.

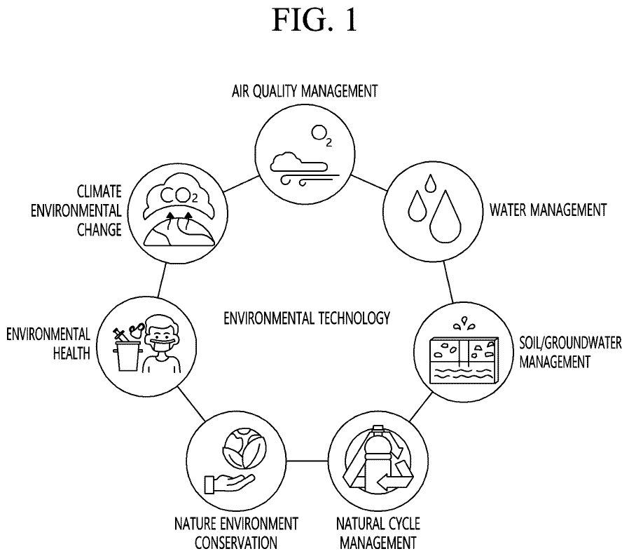

FIG. 1 is a view illustrating the classification system for this environmental technology. With reference to FIG. 1, the environmental technology may be classified into seven technologies (hereinafter referred to as the “seven major environmental technologies”) according to an embodiment. The seven major environmental technologies may include an air quality management technology, a water management technology, a soil and groundwater management technology, a natural cycle management (or waste management) technology, a natural environment conservation technology, an environmental health technology, and a climate environmental change technology.

First, the air quality management technology refers to the various means, methods, and strategies used to monitor, analyze, and control air quality for the protection of public health and the environment. This may include the application of scientific knowledge and engineering principles to understand, evaluate, and manage air pollutants and their sources.

Next, the water management technology refers to various means, methods, and strategies used to maintain and improve the quality of water resources. This may include the application of scientific knowledge and engineering principles to provide safe, clean water while eliminating or minimizing contaminants that occur in the water.

Next, the soil and groundwater management technology refers to various means, methods, and strategies used to maintain and improve the quality of soil and groundwater. This may include the application of scientific knowledge and engineering principles to preserve clean soil and groundwater while reducing or eliminating factors that cause contamination.

Next, the natural cycle management technology is directed to minimizing the consumption and disposal of resources through efficient use and recycling of resources and realizing sustainable resource management. That is, the natural cycle management technology refers to various means, methods, and strategies used to pursue environmental conservation and sustainable development by increasing the effectiveness and utilization of resources and minimizing the loss of resources. This can include the application of scientific knowledge and engineering principles to minimize waste and losses generated in the process of using resources while recovering and recycling used resources.

Next, the natural environment conservation technology refers to various means, methods, and strategies used to maintain ecological balance and biodiversity, and ensure the sustainability of the Earth's ecosystems. This may include the application of scientific knowledge and engineering principles to protect, preserve, and restore the natural environment, promote sustainability, and mitigate the impacts of human activities on ecosystems.

Next, the environmental health technology refers to various means, methods, and strategies to identify, protect, and promote the causal relationship between the environment and human health. This may include the application of scientific knowledge and engineering principles to assess environmental risks and impacts to understand the interaction between the environment and human health, and to promote a clean and safe environment through the monitoring of environmental contamination and pollutant sources to enable healthy living.

Finally, the climate environmental change technology refers to various means, methods, and strategies to solve environmental issues related to climate change and to protect and manage the climate environment. This may include the application of scientific knowledge and engineering principles to understand the composition of the climate environment through the interaction between environmental factors and climate factors, and thereby to respond to changes in the climate environment.

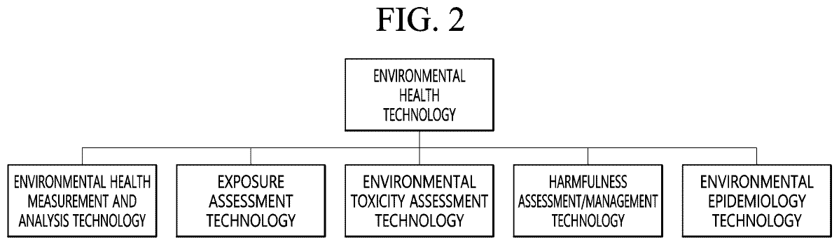

The present invention relates to the environmental health technology, among the environmental technologies. FIG. 2 is a view illustrating detailed technologies of an environmental health technology. With reference to FIG. 2, the environmental health technology may include environmental health measurement and analysis technology, exposure assessment technology, environmental toxicity assessment technology, harmfulness assessment and management technology, and environmental epidemiology technology.

The environmental health measurement and analysis technology refers to a technology that is used to assess and understand the relationship between environmental factors and human health.

An environmental harmful factor measurement and analysis technology may be classified into a hazardous chemical material monitoring technology, an environmental harmful factor exposure amount assessment technology, and the like.

The hazardous chemical material monitoring technology is a technology that measures and monitors the concentration or exposure situation of environmental pollutants, and may include a trace hazardous chemical material sample pretreatment technology, a rapid detection technique, and a monitoring technology for new hazardous chemical materials and other hazardous chemical materials.

The environmental harmful factor exposure amount assessment technology is a technology that measures and assesses the degree of exposure of humans or ecosystems due to environmental pollutants or other harmful factors, and may include an exposure amount assessment technology for physical environmental harmful factors (magnetic waves, noise/vibration, etc.), biological harmful factors (infectious pathogens, etc.), particulate harmful factors (fine dust, microplastics, etc.), gaseous harmful factors, other environmental harmful factors, and the like.

An environmental harmful factor behavior assessment technology may be classified into an environmental harmful factor behavior modeling technique, an environmental harmful factor behavior modeling an environmental harmful factor life cycle assessment technique, and the like.

The environmental harmful factor behavior modeling technique is a technology that understands and assesses the relationship between human activities and behaviors and exposure to environmental pollutants through time-activity behavior analysis of humans, simulation and modeling techniques, and the like, and may include an emission factor calculation technique, contamination measurement technology, a chemical accident reassessment chemical accident safety management technology, an exposure concentration prediction technique, and the like.

The environmental harmful factor behavioral modeling refers to a mathematical modeling technique that quantifies and predicts the correlation between activities and behaviors of humans and exposure to environmental pollutants, and may include technologies such as an environmental media behavioral assessment technique, a multi-media monitoring and follow-up technique, a behavioral modeling technique, and big data machine learning.

The environmental harmful factor life cycle assessment technology is a technology that assesses the life cycle of the generation, emission, migration, exposure, and impact of a specific environmental harmful factor, and may be comprised of a linkage of several assessment techniques such as exposure assessment and harmfulness assessment.

A biological sample analysis and use-based technology may be classified into a biological sample monitoring technology and a bioaccumulation assessment technology.

The biological sample monitoring technology is a technology that measures the degree to which the human body and living organisms are exposed to environmental pollutants or other environmental harmful factors, and may include an exposure monitoring technology using biological samples such as blood/urine (including new human-derived biological samples), a unit technology for simultaneous multi-component high sensitivity biological monitoring, a quality assurance and proficiency testing technology, a biological sample storage and management technology, other biological monitoring-related technologies, and the like.

The bioaccumulation assessment technology is a technology that assesses the degree of accumulation of environmental pollutants or environmental harmful factors in the human body and living organisms, and may include technologies such as assessment of the accumulative potential of environmental harmful factors in living organisms due to short- and long-term exposure to environmental harmful factors, development of measurement methodology for trace harmful factors in living organisms, and assessment of the migration pathways of environmental harmful factors by nutrient levels.

In addition, the environmental health measurement and analysis technology is a technology designed to measure environmental risk factors and analyze their effects on human health, and may include all technologies that investigates the relationship between the environment and human health and provides accurate information to contribute to policy establishment and environmental conservation.

Meanwhile, the exposure assessment technology is a technology that assesses the degree to which humans are exposed to environmental pollutants, and may include all technologies related to exposure assessment, such as identifying the concentration and exposure pathways of environmental pollutants that affect human health.

For example, the exposure assessment technology may include a human body exposure and harmfulness assessment model, exposure modeling (ECETOC TRA, Stoffenmanager, etc.), living environment access media harmfulness assessment, a region-based exposure assessment unit technology, ecological exposure, microcosm, mesocosm, population probabilistic assessment, and the like.

An environmental toxicity assessment technology may include all technologies related to environmental toxicity assessment, such as assessing harmful effects on living organisms by assessing the effects of substances in the environment on ecosystems and living organisms and contributing to the preservation of the environment, the protection of human health, and the like.

For example, the environmental toxicity assessment technology may include a survival rate indicator (Methylthiazol tetrazolium, etc.), a cell death indicator (Annexin V-FITC/PI, etc.), a gene expression analysis technology, a toxicity prediction modeling system, an acute and chronic toxicity assessment, mixed toxicity, a biomarker, a biosensor, an endocrine disruption effect assessment technology, a non-linear toxicity assessment technology, a toxicant identification evaluation (TIE), toxicant reduction evaluation (TRE), an omics integrated database system, in silico, a quantitative structure activity relationship (QSAR) system, national ambient monitoring stations (NAMs), adverse outcome pathway (AOP), integrated approaches to testing and assessment (IATA), in vitro toxicity testing (ITS), reference dose method (RfD method), and the like.

The harmfulness assessment and management technology is a technology that assesses the impact of environmental pollutants on human health and establishes appropriate management and response measures accordingly, and may include all technologies related to harmfulness assessment and management, such as assessing health risks caused by environmental pollutants and developing response measures.

For example, the harmfulness assessment and management technology may include a harmful substance database system, a reference dose method (RfD method), a harmfulness assessment technology using 3D printed materials, a predicted environmental concentration (PEC) assessment technology, a relative risk assessment technology, an analytical technique using species sensitivity distribution (SSD), and the like.

The environmental epidemiology technology may be classified into health impact correlation characterization technology, environmental health and biometric information big data-based technology, health impact prevention and management service technology, and the like.

The health impact correlation characterization technology may be classified into environmental epidemiological investigation technology, exposure, impact, and susceptibility indicator analysis technology, health impact assessment technology for sensitive and highly exposed groups, environmental disease forensic technology, environmental disease prediction technology, and the like.

The environmental epidemiological investigation technology is a technology that enables the understanding and analysis of physical, chemical, and biological phenomena occurring in the environment to assess the impact of environmental contamination on the health of the human body or ecosystems, and may include technologies related to cross-sectional studies, longitudinal studies, cohort randomized clinical trials, panels, intervention studies, and the like.

The exposure, impact, and susceptibility indicator analysis technology is a technology that enables the assessment of whether the human body or ecosystems are exposed to environmental contaminants, how they are affected by those contaminants, and how sensitive they respond, and may include technologies related to harmful impact biomarkers, exposure biomarkers, impact biomarkers, susceptibility biomarkers, multiple exposures, (multi-) omics, and the like.

The health impact assessment technology for sensitive and highly exposed groups is a technology that enables the assessment of the impacts of environmental contaminants on populations and ecosystems that are likely to be exposed to environmental contaminants, and may include health impact assessment-related technologies for sensitive groups, mother-fetus/infants, youth, older populations, generational transitions, underlying diseases, and highly exposed groups.

The environmental disease forensic technology is a technology that enables the identification of the relationship between environmental contamination and human health and the investigation of which environmental contaminants or harmful substances may have caused an environmental disease, and may include technologies related to isotopes, environmental diseases, exposure source tracking, historical exposures, areas of harmfulness concerns, backtracking of exposure amounts, disease contribution of exposure, and identification and tracking of environmental hazardous factors.

The environmental disease prediction technology is a technology that enables the understanding of the relationship between environmental contamination and the health of the human body and the prediction of how specific environmental conditions or hazardous substances may affect the occurrence of environmental diseases, and may include technologies related to environmental disease burden, prediction models, environmental health indicators, and the like.

The environmental health and biometric information big data-based technology may be classified into biobank management technology (sample-linked data), biometric big data utilization technology, big data-platform utilization technology, and the like.

The biobank management technology (sample-linked data) may include technologies related to human body resource banks, linked data, biological sample storage and management, and the like.

The biometric big data utilization technology is a technology that enables the analysis of the interaction between the environment and health using big data, and may include all technologies that use biometric information and big data, such as identifying the correlation between environmental contamination and health problems, establishing monitoring of infectious diseases and early warning systems for environmental diseases, and conducting personalized environmental exposure assessment using big data.

The health impact prevention and management service technology may be classified into health impact prediction and prevention technology, health impact management service technology, and the like.

The health impact prediction and prevention technology is a technology that enables the prediction and prevention of health impacts caused by environmental changes or contamination in advance, and may include technologies related to environmental health big data, health impact prediction, customized preventive care, living labs, and the like.

The health impact management service technology is a technology that monitors the interaction between the environment and health, and effectively manages environmental impacts on health, and may include technologies related to vulnerable groups and vulnerable areas, environmental health services, consumer customization, health impact reduction, and the like.

In addition, the environmental health technology may further include a number of other technologies that can protect human health and ecosystem health from environmental factors, and these technologies may be used to assess environmental risks and impacts, and monitor environmental contamination and contamination sources to ensure a clean and safe environment for healthy living.

Hereinafter, a robot surface disinfection apparatus, a robot surface disinfection method using the same, and a robot surface disinfection system related to the present invention will be described in more detail with reference to the drawings.

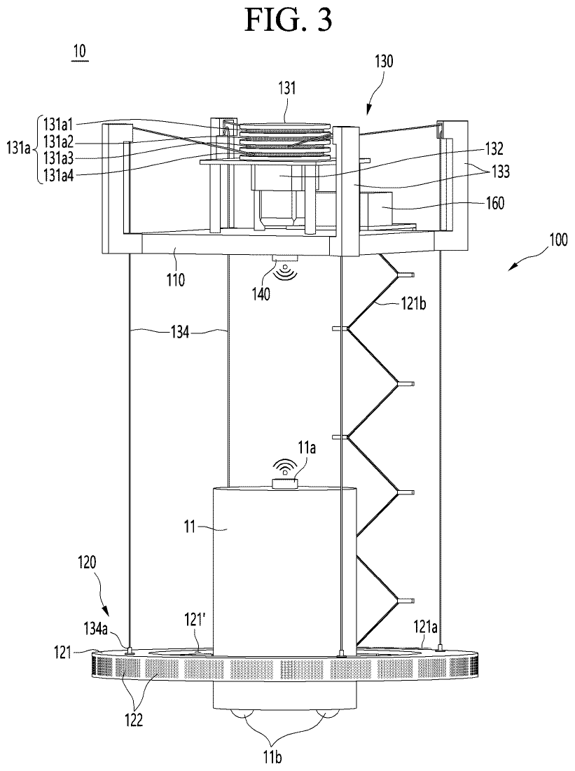

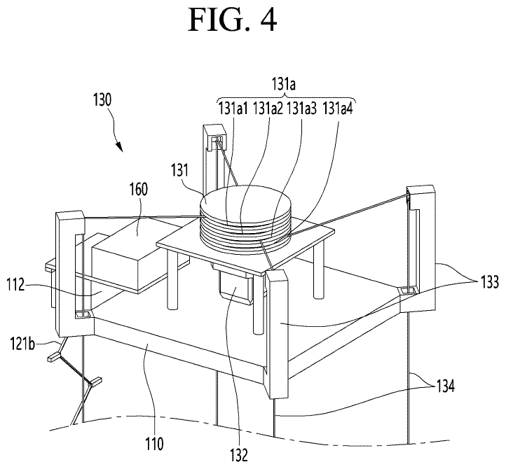

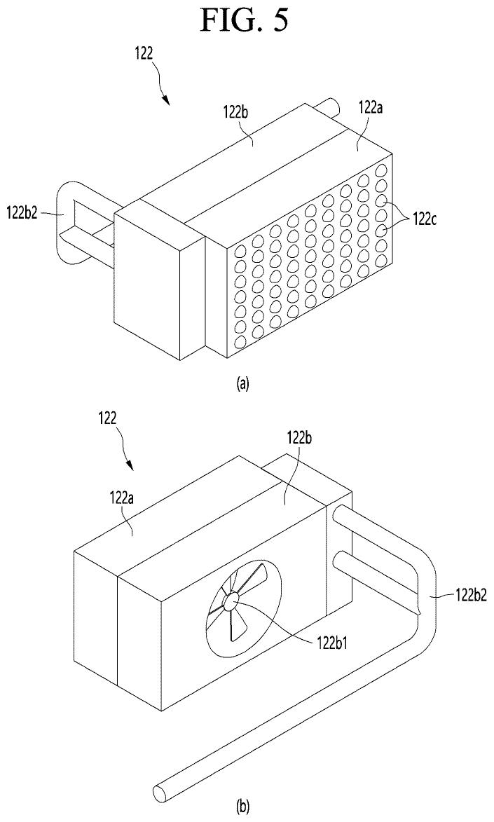

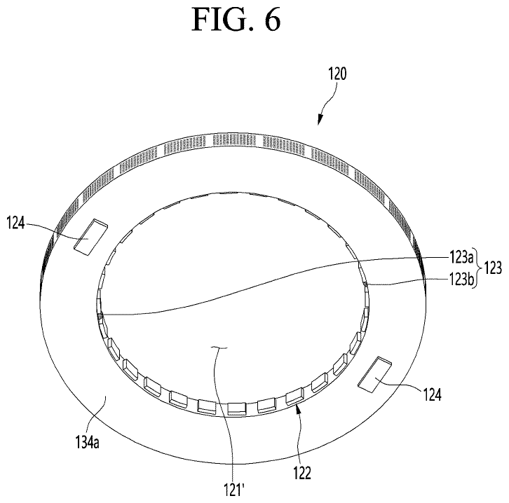

FIG. 3 is a perspective view of a robot surface disinfection apparatus 100 and a robot surface disinfection system 10 including the same, according to an embodiment of the present invention. FIG. 4 is an enlarged perspective view illustrating a lifting module 130 illustrated in FIG. 3. FIG. 5 is a perspective view of an ultraviolet light generating device 122 of a disinfection module 120 illustrated in FIG. 3, viewed from different directions. FIG. 6 is a perspective view viewed from a bottom surface of the disinfection module 120 illustrated in FIG. 3.

With reference to FIGS. 3 to 6, a robot surface disinfection system 10 includes a robot 11 and a robot surface disinfection apparatus 100. The robot surface disinfection system 10 and robot surface disinfection apparatus 100 of the present invention may be used not only for the robot 11, but also for other objects or devices requiring surface disinfection.

The robot 11 may be configured to enable autonomous driving. The bottom surface of the robot 11 facing the ground may be provided with a driving means 11b for autonomous driving of the robot 11. The driving means 11b may be made up of wheels, for example, and may be provided in plurality. Here, the robot 11 may be provided with a plurality of legs to be walkable, such as a quadrupedal or bipedal robot.

The robot surface disinfection apparatus 100 includes a base 110, a disinfection module 120, and a lifting module 130.

The base 110 is positioned at a predetermined height above the ground. The base 110 may be configured to have a built-in structure that is embedded in an upper space of the ceiling of a space in which the robot surface disinfection apparatus 100 is installed.

The disinfection module 120 is formed to be movable up and down between the ground and the base.

A lifting module 130 is connected to the base 110 and the disinfection module 120, respectively, and is configured to lift the disinfection module 120. A direction in which the disinfection module 120 is lifted may be in an upward or downward direction perpendicular to the ground.

The disinfection module 120 may include a hollow frame 121 and an ultraviolet light generating device 122.

The hollow frame 121 defines an exterior appearance of the disinfection module 120, and is provided with a hollow portion 121′ that is open vertically on an inner side thereof. The hollow frame 121 may be formed to have the form of a circular ring, for example, as illustrated in FIG. 3. When the hollow frame 121 has the form of a circular ring, it is characterized in that a distance between a center of the hollow portion 121′ and specific points on an inner circumferential surface of the hollow frame 121 is relatively constant in size and does not vary as the specific points on the inner circumferential surface of the hollow frame 121 vary.

However, the form of the hollow frame 121 is not limited to a circular ring shape, and may be formed to have a polygonal structure, such as a triangle, square, or pentagon having the hollow portion 121′.

In addition, a cable 121b for supplying power to the disinfection module 120 may be connected to the hollow frame 121 and may be provided with a cable housing 121a formed to enable the cable 121b to be received. In addition, the cable 121b may be formed in a foldable manner, as illustrated in FIG. 3, so that the cable 121b may be disposed at least partially overlapping as the disinfection module 120 moves up and down. The base 110 may also be provided with a cable housing 112 in which a cable is received on an opposite side of the cable 121b.

The ultraviolet light generating device 122 may be arranged along the circumference of the hollow frame 121. In the drawings of the present invention, the ultraviolet light generating device 122 is shown to be transversely arranged in a single tier. However, the plurality of ultraviolet light generating devices 122 may be transversely arranged in multiple tiers rather than single tiers, and the number of tiers may be designed to vary. The ultraviolet light generating device 122 is configured to emit ultraviolet light (ultraviolet rays, UV rays) into the hollow portion 121′ of the hollow frame 121 to disinfect a surface of the robot 11 positioned in the hollow portion. Here, ultraviolet light is light with a shorter wavelength than visible light and has a higher energy to enable chemical reactions to take place. Ultraviolet light may destroy the DNA of bacteria with its intense energy, leading to sterilization.

The ultraviolet light generating device 122 may include, for example, a UV-C LED that emits UV-C ultraviolet light as an ultraviolet light source for disinfection. Here, UV-C refers to the relatively short wavelengths of ultraviolet light in the 200 to 280 nm range that have a bactericidal effect on bacterial fungi and microorganisms. UV-C exhibits a high level of bactericidal effect against most bacteria or viruses. In particular, the UV-C LED may select only a specific area without changing physical properties to perform intense sterilization, thus providing a suitable characteristic for surface sterilization.

In addition, the ultraviolet light generating device 122 may be provided with a light generating unit 122a that generates ultraviolet light, and a cooling unit 122b configured to cool the light generating unit 122a, as illustrated in FIG. 5. A cooling method of the cooling unit 122b may be an air-cooled type, a water-cooled type, or a combination thereof. In case of air-cooled type, the cooling unit 122b may be provided with a fan 122b1 formed to perform a heat dissipation function. In case of water-cooled type, the cooling unit 122b may be provided with a circulation tube 122b2 that forms a circulation flow path of cooling water. The circulation tube 122b may be formed of a metallic material or a non-metallic material, and may be configured to be at least partially formed of a resilient material to enable bending.

In addition, the ultraviolet light generating device 122 may be provided with a lens 122c for collecting or dispersing ultraviolet light. Here, the ultraviolet light generating device 122 may be configured such that an irradiation angle and/or irradiation distance of ultraviolet light through the lens 122c is adjustable.

The robot surface disinfection apparatus 100 may be positioned in a state in which the disinfection module 120 is elevated to a height of the robot 11 or higher by the lifting module 130 so that the disinfection module 120 does not act as an obstacle to the movement of the robot 11 when the robot 11 drives the ground to move to a position corresponding to the hollow portion 121′ of the hollow frame 121. In this case, when the disinfection module 120 is elevated to the height of the robot 11 or higher, a completely empty space may be formed between the disinfection module 120 and the ground. Accordingly, the robot 11 may stably move along the ground with no obstruction occurring when moving from the outer side of the disinfection module 120 to a position corresponding to the hollow portion 121′ of the hollow frame 121 through a driving motion or the like.

Then, when the robot 11 enters underneath the disinfection module 120 elevated by the lifting module 130 and moves to a position corresponding to the hollow portion 121′ of the hollow frame 121, the disinfection module 120 may again be lowered by the lifting module 130 to proceed with disinfection of the robot 11 in a state in which the disinfection module 120 is disposed to surround the robot 11.

Meanwhile, the disinfection module 120 may be configured to continuously emit ultraviolet light to the surface of the robot 11 at different heights between the upper end and the lower end of the robot 11 while being lifted by the lifting module 130 in a state in which the robot 11 is positioned in the hollow portion 121′ of the hollow frame 121.

Meanwhile, the disinfection module 120 may further include robot detection sensors 123.

The robot detection sensors 123 may be disposed in the hollow frame 121 facing each other to detect whether the robot 11 is positioned in the hollow portion 121′ of the hollow frame 121.

The robot detection sensors 123 may include a light emitter 123a and a light receiver 123b disposed facing each other, as illustrated in FIG. 6. The light emitter 123a may be configured to generate light toward the light receiver 123b, and the light receiver 123b may be configured to detect the light generated by the light emitter 123a. Further, it may be determined whether the robot 11 is positioned in the hollow portion 121′ based on whether light generated by the light emitter 123a is detected by the light receiver 123b.

For example, it may be determined that the robot 11 is positioned in the hollow portion 121′ when light generated by the light emitter 123a is not detected by the light receiver 123b. In contrast, it may be determined that the robot 11 is not positioned in the hollow portion 121′ when light generated by the light emitter 123a is detected by the light receiver 123b.

Here, the ultraviolet light generating device 122 may be configured to be driven when the robot 11 is detected by the robot detection sensors 123 and not driven when the robot 11 is not detected by the robot detection sensors 123. Accordingly, the disinfection module 120 may be configured to be driven to perform the disinfection function when there is the robot 11 at a height at which the disinfection module 120 is positioned, and not driven when there is no robot 11 at a height at which the disinfection module 120 is positioned. As a result, unnecessary drive of the disinfection module 120 in positions where the robot 11 is not present can be avoided.

Meanwhile, the disinfection module 120 may further include a ground detection sensor 124.

The ground detection sensor 124 may be installed in a bottom portion of the hollow frame 121 that faces the ground. Here, the ultraviolet light generating device 122 may be configured to be not driven when the ground is detected by the ground detection sensor 124. The ground detection sensor 124 may be provided in plurality, and at least some thereof may be disposed in positions symmetrical to each other in the bottom portion of the hollow frame 121.

The ground detection sensor 124 may be configured to be in non-contact with the ground. For example, the ground detection sensor 124 may be configured to detect a position relative to the ground by emitting laser light toward the ground and receiving light reflected back from the ground. Accordingly, the ground detection sensor 124 may detect whether a spacing distance between the hollow frame 121 and the ground falls within a preset range. Alternatively, the ground detection sensor 124 may be configured to be a limit switch that physically contacts the ground to detect a position of the ground.

In addition, the lifting module 130 may be configured to stop the lowering operation when the ground is detected by the ground detection sensor 124. Accordingly, the disinfection module 120 may be prevented from colliding with the ground during the lowering operation. In addition, when the robot 11 is positioned in a lower portion of the hollow frame 121 other than the hollow portion 121′ as well as the ground, the robot 11 may be detected by the ground detection sensor 124, and the lowering operation of the lifting module 130 may be stopped to prevent a collision of the disinfection module 120 with the robot 11 in advance.

Meanwhile, the lifting module 130 may include a bobbin 131, a drive motor 132, a guide bar 133, and a plurality of wires 134.

The bobbin 131 is rotatably installed on the base 110 and may be provided with a plurality of wire windings 131a for each height. For example, the wire windings 131a may be provided with a first wire groove 131a1, a second wire groove 131a2, a third wire groove 131a3, and a fourth wire groove 131a4.

The drive motor 132 may be formed to rotate the bobbin 131. The drive motor 132 may be an electric motor.

The guide bar 133 may be installed in a plurality of places on the base 110. The guide bar 133 may be disposed in a peripheral area of the base 110, respectively, as illustrated in FIG. 2. As an example of the guide bar 133, the drawings of the present invention show the guide bar 133 comprising four guide bars disposed in symmetrical positions with respect to each other in the peripheral area of the base 110.

The wires 134 may be wound on the plurality of wire windings 131a, respectively, and configured to be connected to the hollow frame 121 through the guide bar 133. The hollow frame 121 may be provided with a wire fixing member 134a for fixing one end of the wire 134 being connected to the hollow frame 121.

Here, the plurality of wires 134 may be unwound from the plurality of wire windings 131a or wound onto the plurality of wire windings 131a, by forward or reverse rotation of the drive motor 132, to lift the disinfection module 120.

Meanwhile, in order to guide the robot 11 into a predetermined area, the robot surface disinfection system 10 may further include an infrared light receiving module 11a. In addition, the robot surface disinfection apparatus 100 may further include an infrared light transmitting module 140 as a configuration corresponding to the infrared light receiving module 11a.

The infrared light receiving module 11a may be configured to detect an infrared signal present in the surroundings.

The infrared light transmitting module 140 may be configured to guide the robot 11 by transmitting an infrared signal to an area wider than the predetermined area to which the robot 11 is guided. The infrared light transmitting module 140 may be installed on the bottom surface of the base 110, as illustrated in FIG. 3, to emit infrared light in a downward direction toward the ground.

According to the robot surface disinfection apparatus 100 as described above, normally, the disinfection module 120, which disinfects the surface of the robot 11 by emitting ultraviolet light to the robot 11, and the other major configurations of the robot surface disinfection apparatus 100 are disposed at a position away from the ground at a predetermined heigh, and only move closer to the ground while the disinfection module 120 is performing the disinfection function. Accordingly, the robot surface disinfection apparatus 100 does not act as an obstacle to the movement of the robot 11 to be disinfected, particularly of the autonomous driving robot 11, so that the process of disinfecting the surface of the robot 11 may be accomplished more quickly, and the problem of reduced utilization of a space in which the robot surface disinfection apparatus 100 is installed may be solved to a large extent.

In addition, the disinfection module 120 forms an ultraviolet light irradiation area throughout the hollow portion 121′ of the hollow frame 121 by the ultraviolet light generating device 122 arranged along the circumference of the hollow frame 121. Accordingly, disinfection of the surface of the robot 11 may be more effectively performed in a relatively simple operation in which the disinfection module 120 moves up and down while the robot 11 is positioned in the hollow portion 121′.

Meanwhile, although not illustrated in this drawings, the robot surface disinfection system 10 may further include a sensor configured to detect the movement of an object around the robot surface disinfection apparatus 100 or whether a door of a room in which the robot surface disinfection apparatus 100 is positioned (e.g., a disinfection room) operates. A control unit 160 of the robot surface disinfection apparatus 100 may be configured to control a drive of the disinfection module 120 on the basis of a result detected by the sensor.

For example, when a human entry into a predetermined area around the robot surface disinfection apparatus 100 is detected by the sensor while the disinfection module 120 is being driven, the control unit 160 of the robot surface disinfection apparatus 100 may switch the disinfection module 120 to a non-driven state on the basis of the detection result. Alternatively, when the door opening of the disinfection room in which the robot surface disinfection apparatus 100 is positioned is detected by the sensor while the disinfection module 120 is being driven, the control unit 160 of the robot surface disinfection apparatus 100 may switch the disinfection module 120 to a non-driven state on the basis of the detection result.

Accordingly, exposure of humans to ultraviolet light generated by the disinfection module 120 may be prevented.

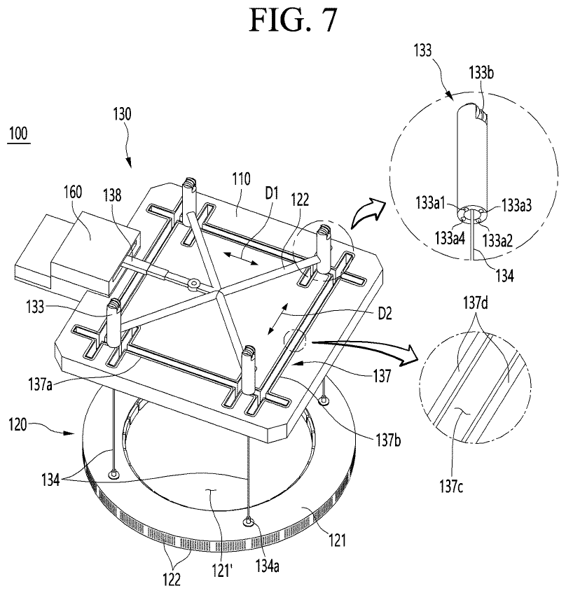

Hereinafter, another example of the lifting module 130 will be described with reference to FIG. 7.

FIG. 7 is a perspective view illustrating another example of the lifting module 130 illustrated in FIG. 3.

With reference to FIG. 7, the lifting module 130 may further include a connecting frame 136, a rail portion 137, and a drive unit 138.

The connecting frame 136 is configured to connect the guide bars 133 to each other to fix relative positions of the guide bars 133 with respect to each other.

The rail portion 137 may be formed to extend in an intersecting shape to guide the movement of the guide bar 133, so that the guide bar 133 is movable in a first direction D1 and a second direction D2, respectively, that are perpendicular to each other. The rail portion 137 may comprise a first rail 137a to guide movement in the first direction D1 and a second rail 137b to guide movement in the second direction D2.

The drive unit 138 may be configured to move the connecting frame 136 in the first direction D1 or the second direction D2.

The wire 134 may be configured to pass through the guide bar 133 and the rail portion 137 to connect to the hollow frame 121. Meanwhile, with reference to FIG. 7, each of the plurality of wires 134 may be configured to be adjustable in length by being wound or unwound from one end of the guide bar 133 by a motor 133b installed on each of the guide bars 133, unlike in FIG. 4.

Meanwhile, the rail portion 137 may be provided with a communication hole 137c and a guide groove 137d.

The communication hole 137c is configured to allow the wire 134 to pass through.

The guide groove 137d are formed on both sides of the communication hole 137c, enabling rolling contact of balls 133a1, 133a2, 133a3, and 133a4 provided at the lower portion of the guide bar 133.

For example, the balls 133a1, 133a2, 133a3, and 133a4 may be configured as first to fourth balls 133a1, 133a2, 133a3, and 133a4 provided on a bottom surface of each of the guide bars 133 and formed to be in rolling contact with the rail portion 137.

The first to fourth balls 133a1, 133a2, 133a3, and 133a4 may be disposed spaced apart from each other at 90 degree intervals along the circumference of the bottom surface of the guide bar 133. In addition, the first and second balls 133a1 and 133a2, and the third and fourth balls 133a3 and 133a4, respectively, may be disposed symmetrically with respect to each other with a center of the bottom surface of the guide bar 133 interposed therebetween. Here, the first and second balls 133a1 and 133a2 may be configured to rotate along a guide groove 137d provided in the first rail 137a when the guide bar 133 moves in the first direction D1, and the third and fourth balls 133a3 and 133a4 may be configured to rotate along a guide groove 137d provided in the second rail 137b when the guide bar 133 moves in the second direction D2. The first to fourth balls 133a1, 133a2, 133a3, and 133a4 may be made of a ball plunger. According to the structure of the lifting module 130 as described above, the disinfection module 120 may be moved a predetermined distance in a horizontal direction with respect to the ground. Accordingly, when the robot 11 is positioned away from a predetermined area of the hollow portion 121′ of the hollow frame 121, the disinfection module 120 may be subtly moved and disposed so that the robot 11 is positioned in a predetermined area of the hollow portion 121′ of the hollow frame 121.

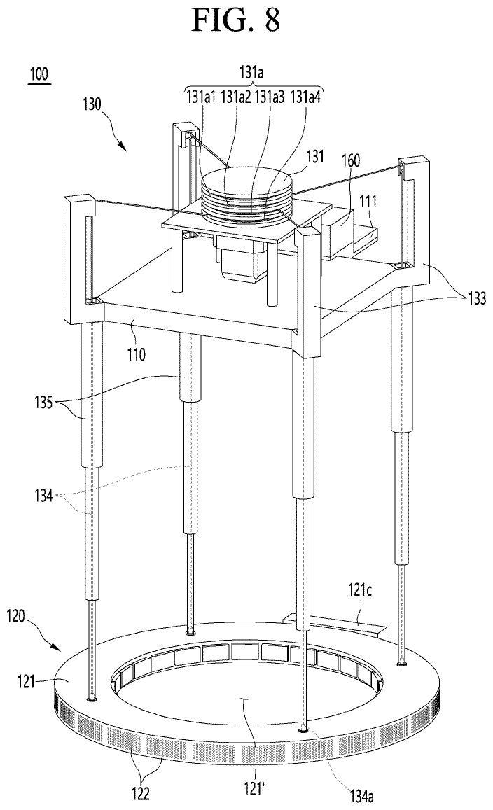

Hereinafter, another example of the robot surface disinfection apparatus 100 will be described with reference to FIG. 8.

FIG. 8 is a perspective view illustrating another example of the robot surface disinfection apparatus 100 illustrated in FIG. 1.

With reference to FIG. 8, the lifting module 130 may further include a multi-stage telescopic cylinder 135.

The multi-stage telescopic cylinder 135 is connected to the base 110 and the hollow frame 121, respectively, and is formed to surround the wire 134 therein, but may be formed to be extendable or contractible by length adjustment of the wire 134. That is, the multi-stage telescopic cylinder 135 may be configured as a plurality of cylinders that are formed to be mutually insertable, and may be adjustable in length so as to at least partially overlap or be released from the overlapping state. Accordingly, the lifting operation of the disinfection module 120 may be more stable and without shaking, compared to when the wire 134 is formed to be exposed to the outside. Further, the wire 134 may be disposed to be surrounded inside the multi-stage telescopic cylinder 135, thereby preventing damage to the wire 134.

Meanwhile, a battery 121c may be installed in the hollow frame 121 to supply power to the ultraviolet light generating device 122. In addition, a wireless charger 111 may be installed on the base 110 to wirelessly charge the battery 121c when the disinfection module 120 is disposed adjacent to the base 110 within a predetermined distance by the lifting module 130. Accordingly, the configuration of the cable 121b for supplying power to the disinfection module 120 may be excluded, which may lead to implementing a more simplified configuration of the robot surface disinfection apparatus 100 and the robot surface disinfection system 10. Meanwhile, the wireless charger 111 provided in the base 110 may be disposed in a single housing together with the control unit 160. The control unit 160 may be configured to perform overall control functions for major configurations of the robot surface disinfection apparatus 100 and the robot surface disinfection system 10.

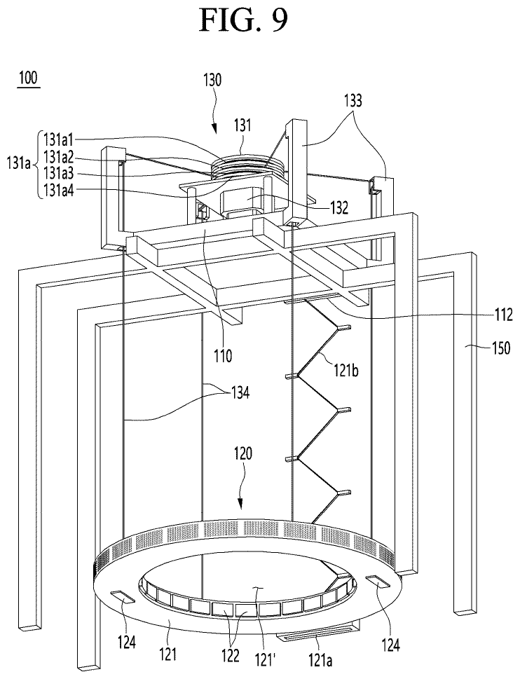

Hereinafter, still another example of the robot surface disinfection apparatus 100 will be described with reference to FIG. 9.

FIG. 9 is a perspective view illustrating still another example of the robot surface disinfection apparatus 100 illustrated in FIG. 3.

With reference to FIG. 9, the robot surface disinfection apparatus 100 may be configured to be movable. The robot surface disinfection apparatus 100 may further include a mobile frame 150.

The mobile frame 150 may be formed to extend in a direction perpendicular to the ground. Here, the base 110 may be fixedly installed on an upper portion of the mobile frame 150. Accordingly, the disinfection module 120 may be configured to move with the base 110 as a position of the mobile frame 150 is moved. As a result, the robot surface disinfection apparatus 100 may move to a position where disinfection of the robot 11 is required and proceed to disinfect the surface of the robot 11.

A lower end of the mobile frame 150 may be configured in a form in which a lower end of the mobile frame 150 is not provided with a separate transportation means as illustrated in FIG. 9. Although not illustrated in the drawings of the present invention, the lower end of the mobile frame 150 may be provided with a transportation means for moving the mobile frame 150. The transportation means may be, for example, wheels.

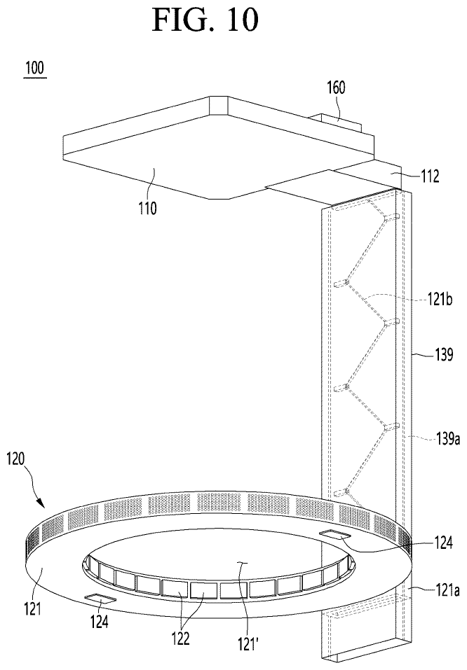

Hereinafter, still another example of the robot surface disinfection apparatus 100 will be described with reference to FIG. 10.

FIG. 10 is a perspective view illustrating still another example of the robot surface disinfection apparatus 100 illustrated in FIG. 3.

With reference to FIG. 10, the lifting module 130 may include a rail plate 139.

The rail plate 139 may be formed to extend in a direction perpendicular to the ground as illustrated in FIG. 10. The rail plate 139 may be disposed to be fixed to a wall of a space in which the robot surface disinfection apparatus 100 is installed.

An empty space may be formed on the interior of the rail plate 139. The cable 121b that supplies power to the disinfection module 120 may be received in an empty space inside the rail plate 139.

A guide rail 139a may be formed on the rail plate 139 to guide the up and down movement of the disinfection module 120 along a length direction of the rail plate 139 extending upward and downward. Here, the cable housing 121a, which is formed on one side of the hollow frame 121 of the disinfection module 120, is seated on the guide rail 139a and may be moved along the guide rail 139a with the hollow frame 121. The cable housing 121a may be configured to receive the cable 121b for supplying power to the disinfection module 120.

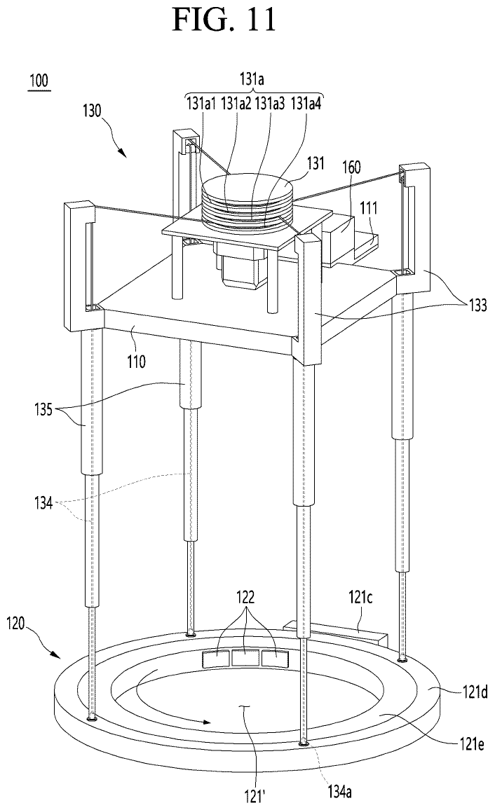

Hereinafter, still another example of the robot surface disinfection apparatus 100 will be described with reference to FIG. 11.

FIG. 11 is a perspective view illustrating still another example of the robot surface disinfection apparatus 100 illustrated in FIG. 3.

With reference to FIG. 11, the hollow frame 121 includes a first frame 121d and a second frame 121e configured to be rotatable relative to each other. The second frame 121e may be coupled to the inner side of the first frame 121d so as to be rotatable relative to the first frame 121d. For the relative rotation described above, the first and second frames 121d and 121e may have the form of a concentric ring.

The ultraviolet light generating device 122 is installed in at least one area of the circumference of the inner side of the second frame 121e, and is configured to emit ultraviolet light into the hollow portion 121′, which is a space in the inner side of the second frame 121e. As the second frame 121e rotates relative to the first frame 121d, the ultraviolet light generating device 122 will emit ultraviolet light while rotating 360 degrees around the robot 11 positioned in the hollow portion 121′.

The first frame 121d is connected to the lifting module 130. Accordingly, the first frame 121d and the second frame 121e coupled thereto are configured to be elevated or lowered by the lifting module 130.

The first frame 121d is configured to allow a lifting operation by the lifting module 130, but not to rotate with the second frame 121e during the rotation operation of the second frame 121e, as described below. As described above, since the first frame 121d is configured to only perform a lift operation and not rotate, configurations that are connected to the lifting module 130 for the lifting operation of the disinfection module 120 may be disposed in the first frame 121d to prevent configurations such as the wires 134 or the multi-stage telescopic cylinder 135 for the connection of the disinfection module 120 and the lifting module 130 from becoming tangled during the rotation operation of the disinfection module 120 or otherwise interfering with the rotation operation of the disinfection module 120.

The second frame 121e is disposed on the inner side of the first frame 121d and is connected to the first frame 121d so as to be rotatable thereto around the hollow portion 121′ of the hollow frame 121. Further, the second frame 121e is formed to be liftable along with the first frame 121d by the lifting module 130 while connected to the first frame 121d. As described above, the second frame 121e is configured such that not only the lifting operation by the lifting module but also the rotating operation around the hollow portion 121′ may be performed.

The relative rotation operation of the second frame 121e may be accomplished by the power transmission unit (not illustrated). The power transmission unit may be installed within the hollow frame 121.

For example, the power transmission unit may be disposed between the first and second frames 121d and 121e and may include a plurality of gears. Further, for smooth relative rotation, rollers, balls, and the like may be provided between the first frame 121d and the second frame 121e, respectively, in rolling contact therewith.

The power transmission unit may be supplied with power in a wired manner through a power cable connected through the multi-stage telescopic cylinder 135. Alternatively, the power transmission unit may be supplied with power in a wireless manner, such as from the battery 121c installed in the hollow frame 121 and made rechargeable by the wireless charger 111. The battery 121c may be configured to be charged by receiving power wirelessly from the wireless charger 111 in a state in which the disinfection module 120 is disposed adjacent to the base 110 within a predetermined distance by the lifting module 130.

Further, the ultraviolet light generating device 122 may also be configured to be supplied with power in a wireless manner. To this end, the second frame 121e or ultraviolet light generating device 122 may be provided with a wirelessly rechargeable auxiliary battery (not illustrated). To implement the same charging manner as the wireless charger 111-battery 121c described above, the base 110 may be provided with a wireless charger (not illustrated) for charging the auxiliary battery.

In contrast, the ultraviolet light generating device 122 may be configured to be supplied with power in a wired manner. To this end, a power supply rail (not illustrated), which is connected to a power cable, may be disposed in the form of a loop on the inner circumferential surface of the first frame 121d. In addition, a power receiving rail (not illustrated) may be disposed in the form of a loop on the outer circumferential surface of the second frame 121e, the power receiving rail maintaining a state of being in contact with the power supply rail at all times and receiving power, even when rotating relative to the power supply rail, and being electrically connected to the ultraviolet light generating device 122. That is, the power supply rail and power receiving rail are formed in the form of a ring and disposed facing each other.

The ultraviolet light generating device 122 may be arranged along the circumference of the second frame 121e, but may be disposed only on a portion of the circumference of the second frame 121e as illustrated in FIG. 11. The ultraviolet light generating device 122 may be configured with one or more units. In FIG. 11, the ultraviolet light generating device 122 is shown configured with three units.

For reference, in FIG. 11, the ultraviolet light generating device 122 is illustrated as having the same size and being equally spaced from each other, but is not necessarily limited to this form. For example, the ultraviolet light generating device 122 may be formed to have different sizes, with varying intervals therebetween, and some may be disposed in different forms.

According to the structure described above, the robot surface disinfection apparatus 100 is configured to proceed with disinfection of the robot 11 with the robot 11 surrounded by the disinfection module 120 while the second frame 121e rotates relative to the first frame 121d around the hollow portion 121′ of the hollow frame 121. According to this configuration, the ultraviolet light generating device 122 may perform disinfection across the entire 360-degree area surrounding the robot 11 while rotating along the circumference of the hollow portion 121′.

Accordingly, the number of ultraviolet light generating devices 122 mounted on the hollow frame 121 may be minimized, thereby reducing the power consumption required for operation of the ultraviolet light generating devices 122, and disinfection of the robot 11 may be effectively performed with fewer ultraviolet light generating devices 122.

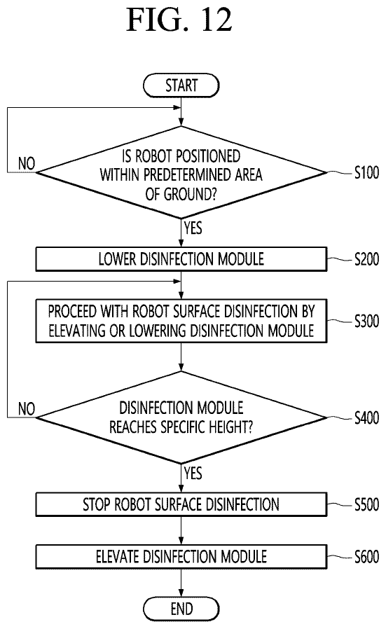

Hereinafter, still another example of the robot surface disinfection apparatus 100 will be described with reference to FIG. 12.

FIG. 12 is a flowchart of a surface disinfection method of the robot 11 using the robot surface disinfection apparatus 100 according to another embodiment of the present invention.

With reference to FIG. 12, a robot surface disinfection method using the robot surface disinfection apparatus 100 may include detecting whether the robot 11 is positioned within a predetermined area of the ground (S100) and, when the robot 11 is detected to be positioned within the predetermined area, driving the robot surface disinfection apparatus 100 to proceed with surface disinfection of the robot 11 (S200 and S300). Here, steps S200 and S300 of proceeding with surface disinfection of the robot 11 may be configured such that the disinfection module 120 continuously emits ultraviolet light at different heights between the upper end and the lower end of the robot 11 while being lifted by the lifting module 130.

In addition, steps S200 and S300 of proceeding with surface disinfection of the robot 11 may include lowering the disinfection module 120 when it is detected whether the robot 11 is positioned within a predetermined area of the ground (S200), and proceeding with disinfection of the surface of the robot 11 while elevating or lowering the disinfection module 120 (S300).

In addition, the robot surface disinfection method using the robot surface disinfection apparatus 100 may further include detecting an upper end and a lower end of the robot 11 to detect whether the disinfection module 120 has reached a specific height (S400), and stopping the surface disinfection of the robot 11 when the disinfection module 120 deviates from a position between the upper end and the lower end of the robot 11 as a result of the detection (S500). In contrast, when it is detected that the disinfection module 120 has reached a specific height (S400) and the disinfection module 120 does not deviate from a position between the upper end and the lower end of the robot 11 as a result of the detection, step 300 of proceeding with disinfection of the surface of the robot 11 may be performed continuously by elevating or lowering the disinfection module 120 without stopping the surface disinfection of the robot 11.

Meanwhile, the robot surface disinfection method using the robot surface disinfection apparatus 100 may further include, after step S500 of stopping the surface disinfection of the robot 11, elevating the disinfection module 120 to return the disinfection module 120 to an initial position before being driven (S600).

What is claimed is:

1. A robot surface disinfection apparatus comprising:
a base positioned at a predetermined height from the ground;
a disinfection module formed to be movable up and down between the ground and the base; and
a lifting module connected to the base and the disinfection module, respectively, and configured to lift the disinfection module,
wherein the disinfection module includes:
a hollow frame provided with a hollow portion that is open vertically; and
an ultraviolet light generating device arranged along a circumference of the hollow frame, and configured to emit ultraviolet light into the hollow portion to disinfect a surface of a robot positioned in the hollow portion, and
wherein, when the robot drives on the ground and moves to a position corresponding to the hollow portion, the disinfection module is positioned in a state elevated to a height of the robot or higher by the lifting module so that the disinfection module does not act as an obstacle to the movement of the robot, and when the robot enters underneath the elevated disinfection module and moves to a position corresponding to the hollow portion, the disinfection module is lowered by the lifting module to proceed to disinfect the robot in a state disposed to surround the robot.

2. The robot surface disinfection apparatus of claim 1, wherein the disinfection module is configured to continuously emit ultraviolet light at different heights between an upper end and a lower end of the robot while being lifted by the lifting module in a state in which the robot is positioned in the hollow portion.

3. The robot surface disinfection apparatus of claim 2, wherein the disinfection module further includes robot detection sensors disposed on the hollow frame facing each other and configured to detect whether the robot is positioned in the hollow portion, and
wherein the ultraviolet light generating device is configured to be driven when the robot is detected by the robot detection sensors and not driven when the robot is not detected by the robot detection sensors.

4. The robot surface disinfection apparatus of claim 3, wherein the disinfection module further includes a ground detection sensor installed at a bottom portion of the hollow frame facing the ground, and
wherein the ultraviolet light generating device is configured to be not driven when the ground is detected by the ground detection sensor.

5. The robot surface disinfection apparatus of claim 4, wherein the lifting module is configured to stop a lowering operation when the ground is detected by the ground detection sensor.

6. The robot surface disinfection apparatus of claim 1, wherein the lifting module includes:
a bobbin rotatably installed on the base and provided with a plurality of wire windings for each height;
a drive motor formed to rotate the bobbin;
guide bars installed at a plurality of places of the base; and
a plurality of wires each wound on the plurality of wire windings and connected to the hollow frame through the guide bars, and
wherein the plurality of wires are configured to be unwound from the plurality of wire windings or wound on the plurality of wire windings by forward or reverse rotation of the drive motor to lift the disinfection module.

7. The robot surface disinfection apparatus of claim 6, wherein the lifting module further includes a multi-stage telescopic cylinder connected to the base and the hollow frame, respectively, and formed to surround the wire therein and extendable or contractable by length adjustment of the wire.

8. The robot surface disinfection apparatus of claim 6, wherein the lifting module further includes:
a connecting frame configured to connect the guide bars to each other to fix relative positions of the guide bars with respect to each other;
a rail portion formed to extend in an intersecting form to guide movement of the guide bars so that the guide bars are movable in a first direction and a second direction, respectively, that are perpendicular to each other; and
a drive unit configured to move the connecting frame in the first direction or the second direction, and
wherein the wire passes through the guide bars and the rail portion to be connected to the hollow frame.

9. The robot surface disinfection apparatus of claim 8, wherein the rail portion includes:
a communication hole configured for the wire to pass through; and
a guide groove formed on both sides of the communication hole, enabling rolling contact of balls provided at a lower portion of the guide bar.

10. The robot surface disinfection apparatus of claim 1, wherein a battery is installed in the hollow frame, configured to supply power to the ultraviolet light generating device, and a wireless charger is installed in the base, configured to wirelessly charge the battery in a state in which the disinfection module is disposed adjacent to the base within a predetermined distance by the lifting module.

11. The robot surface disinfection apparatus of claim 1, wherein the hollow frame includes:
a first frame connected to the lifting module; and
a second frame disposed on an inner side of the first frame and connected to the first frame so as to be rotatable thereto around the hollow portion, and
wherein the ultraviolet light generating device is disposed in at least one area of an inner circumference of the second frame and is configured to emit ultraviolet light toward the hollow portion inside the second frame, and
wherein, when the disinfection module is disposed to surround the robot, disinfection of the robot proceeds while the second frame rotates relative to the first frame around the hollow portion.

12. The robot surface disinfection apparatus of claim 11, wherein a power supply rail connected to a power cable is disposed in the form of a loop on an inner circumferential surface of the first frame, and a power receiving rail electrically connected to the ultraviolet light generating device is disposed in the form of a loop on an outer circumferential surface of the second frame, the power receiving rail always maintaining a state of being in contact with the power supply rail and receiving power even during the relative rotation.

13. A robot surface disinfection method using a robot surface disinfection apparatus, the robot surface disinfection method comprising:
detecting whether a robot is positioned within a predetermined area of the ground; and
proceeding disinfection of a surface of the robot by driving a robot surface disinfection apparatus when the robot is detected to be positioned within the predetermined area,
wherein the robot surface disinfection apparatus includes:
a base positioned at a predetermined height from the ground;
a disinfection module formed to be movable up and down between the ground and the base; and
a lifting module connected to the base and the disinfection module, respectively, and configured to lift the disinfection module,
wherein the disinfection module includes:
a hollow frame provided with a hollow portion that is open vertically; and
an ultraviolet light generating device arranged along a circumference of the hollow frame, and configured to emit ultraviolet light into the hollow portion to disinfect a surface of the robot positioned in the hollow portion within the predetermined area, and
wherein, when the robot drives on the ground and moves to a position corresponding to the hollow portion, the disinfection module is positioned in a state elevated to a height of the robot or higher by the lifting module so that the disinfection module does not act as an obstacle to the movement of the robot, and when the robot enters underneath the elevated disinfection module and moves to a position corresponding to the hollow portion, the disinfection module is lowered by the lifting module to proceed to disinfect the robot in a state disposed to surround the robot.

14. The robot surface disinfection method of claim 13, wherein the proceeding of disinfection of the surface of the robot is configured to continuously emit ultraviolet light, by the disinfection module, at different heights between an upper end and a lower end of the robot while the disinfection module is being lifted by the lifting module.

15. The robot surface disinfection method of claim 14, further comprising:
stopping disinfection of the surface of the robot by detecting the upper end and the lower end of the robot, when the disinfection module deviates from a position between the upper end and the lower end of the robot.

16. A robot surface disinfection system comprising:
a robot configured to enable autonomous driving; and
a robot surface disinfection apparatus configured to disinfect a surface of the robot positioned within a predetermined area of the ground, in communication with the robot,
wherein the robot surface disinfection apparatus includes:
a base positioned at a predetermined height from the ground;
a disinfection module formed to be movable up and down between the ground and the base; and
a lifting module connected to the base and the disinfection module, respectively, and configured to lift the disinfection module,
wherein the disinfection module includes:
a hollow frame provided with a hollow portion that is open vertically; and
an ultraviolet light generating device arranged along a circumference of the hollow frame, and configured to emit ultraviolet light into the hollow portion to disinfect a surface of a robot positioned in the hollow portion, and
wherein, when the robot drives on the ground and moves to a position corresponding to the hollow portion, the disinfection module is positioned in a state elevated to a height of the robot or higher by the lifting module so that the disinfection module does not act as an obstacle to the movement of the robot, and when the robot enters underneath the elevated disinfection module and moves to a position corresponding to the hollow portion, the disinfection module is lowered by the lifting module to proceed to disinfect the robot in a state disposed to surround the robot.

17. The robot surface disinfection system of claim 16, wherein the robot further includes an infrared light receiving module configured to detect an infrared signal, and
wherein the robot surface disinfection apparatus includes an ultraviolet light generating device that further includes an infrared light transmitting module configured to guide the robot by transmitting an infrared signal to an area wider than the predetermined area.

18. The robot surface disinfection system of claim 16, further comprising:
a sensor configured to detect whether an object around the robot surface disinfection apparatus is moving or whether a door in a room in which the robot surface disinfection apparatus is positioned is operating,
wherein a control unit of the robot surface disinfection apparatus controls driving of the disinfection module on the basis of a result detected by the sensor.
